(19) United States
(12) Patent Application Publication
(10) Pub. No.: US 2016/0074123 A1
(43) Pub. Date: Mar. 17, 2016

(54) Surgery Pathway Guidance And Boundary System

(71) Applicants: Randall BLY, Seattle, WA (US); Blake HANNAFORD, Seattle, WA (US); Kris S. MOE, Seattle, WA (US); UNIVERSITY OF WASHINGTON through its CENTER FOR COMMERCIALIZATION, Seattle, WA (US)

(72) Inventors: Randall BLY, Seattle, WA (US); Blake HANNAFORD, Seattle, WA (US); Kris S. MOE, Seattle, WA (US)

(21) Appl. No.: 14/787,107

(22) PCT Filed: May 30, 2014
(86) PCT No.: PCT/US2014/040161

§ 371 Date: Oct. 26, 2015

Related U.S. Application Data
(60) Provisional application No. 61/829,474, filed on May 31, 2013.

(51) Int. Cl. A61B 19/00

(52) U.S. Cl. CPC A61B 19/50; A61B 19/2203; A61B 19/56; A61B 2019/507; A61B 2019/5265; A61B 2019/2292; A61B 2017/00123

(57) ABSTRACT

Described herein are methods and systems for defining surgical boundaries. One example method involves receiving data indicating a representation of a patient; receiving data indicating (i) a surgical target region within the representation of the patient; and (ii) a surgical entry portal within the representation of the patient; providing a graphical display of (i) the representation of the patient, and (ii) a surgical pathway from the surgical entry portal to the surgical target region; defining one or more surgical boundaries within the representation; receiving data indicating a position of a surgical instrument with respect to the representation; based on the received data indicating the position of the surgical instrument, determining that the surgical instrument is within a threshold distance from the one or more surgical boundaries; and providing feedback indicating that the surgical instrument is within the predetermined threshold distance from the one or more surgical boundaries.

### CROSS-REFERENCE TO RELATED APPLICATION

This application claims priority to U.S. Provisional Patent Application Ser. No. 61/829,474 filed May 31, 2013 entitled Endoscopic Surgery Pathway Guidance and Boundary System, which is incorporated herein in its entirety.

### BACKGROUND

Unless otherwise indicated herein, the materials described in this section are not prior art to the claims in this application and are not admitted to be prior art by inclusion in this section.

Surgical navigation systems may assist surgeons in navigation of surgical instruments during surgical procedures, such as endoscopic sinus surgery. Such surgical procedures may involve inserting a surgical instrument into a surgical portal, traversing a surgical pathway from the surgical portal to a surgical target region, and manipulating the surgical target region. Some surgical navigation systems include a display on which the location of the surgical instruments is overlaid onto one or more 2-D representations of the patient (e.g., CT or MRI images). Using this system, the surgeon can identify the location of a surgical instrument, but not whether the surgical instrument is correctly proceeding down the surgical pathway, or whether the surgical instrument is correctly manipulating the surgical target region. As a result, in some instances, a surgical instrument can get “lost” relative to the surgical pathway or the surgical target region. At the same time, precise navigation of the surgical instrument is important, as vital anatomical features may be in close proximity to the surgical pathway or the surgical target region.

### SUMMARY

In one aspect, a computer-implemented method is provided. The method may involve: receiving, by a computing device, data for a patient indicating a representation of the patient; receiving data indicating (i) a surgical target region within the representation of the patient; and (ii) a surgical entry portal within the representation of the patient; providing a graphical display of (i) the representation of the patient, and (ii) a surgical pathway from the surgical entry portal to the surgical target region within the representation; defining one or more surgical boundaries within the representation, receiving data indicating a position of a surgical instrument with respect to the representation of the patient; based on the received data indicating the position of the surgical instrument, determining that the surgical instrument is within a pre-determined threshold distance from at least one of the one or more surgical boundaries; and in response to the determining, providing feedback indicating that the surgical instrument is within the pre-determined threshold distance from at least one of the one or more surgical boundaries.

In another aspect, a computing system is provided. The computing system may include: a physical, non-transitory computer readable medium; and program instructions stored on the physical computer readable medium and executable by at least one processor to cause the computing system to: receive data for a patient indicating a representation of the patient; receive data indicating (i) a surgical target region within the representation of the patient: and (ii) a surgical entry portal within the representation of the patient; provide a graphical display of (i) the representation of the patient, and (ii) a surgical pathway from the surgical entry portal to the surgical target region within the representation; defining one or more surgical boundaries within the representation, receive data indicating a position of a surgical instrument with respect to the representation of the patient; based on the received data indicating the position of the surgical instrument, determine that the surgical instrument is within a pre-determined threshold distance from at least one of the one or more surgical boundaries; and in response to the determining, provide feedback indicating that the surgical instrument is within the pre-determined threshold distance from at least one of the one or more surgical boundaries.

In another aspect, an article of manufacture including a non-transitory tangible computer readable medium is provided. The non-transitory tangible computer readable medium may be configured to store at least executable instructions. The executable instructions, when executed by a processor of a computing device, may cause the computing device to perform functions. The functions may include: receiving data for a patient indicating a representation of the patient: receiving data indicating (i) a surgical target region within the representation of the patient; and (ii) a surgical entry portal within the representation of the patient; providing a graphical display of (i) the representation of the patient, and (ii) a surgical pathway from the surgical entry portal to the surgical target region within the representation; defining one or more surgical boundaries within the representation, receiving data indicating a position of a surgical instrument with respect to the representation of the patient: based on the received data indicating the position of the surgical instrument, determining that the surgical instrument is within a pre-determined threshold distance from at least one of the one or more surgical boundaries; and in response to the determining, providing feedback indicating that the surgical instrument is within the pre-determined threshold distance from at least one of the one or more surgical boundaries.

For example, the methods and systems described herein may be employed during surgical procedures to facilitate guidance of a surgical instrument into a surgical portal, along a surgical pathway from the surgical portal to a surgical target region, and within the surgical target region. In one example, a computing system (such as may be part of a surgical navigation system) may receive a representation of a patient that includes the surgical target region and surgical pathway. A surgeon may define surgical boundaries around portions of the surgical pathway and/or the surgical target region to divide the surgical pathway and/or the surgical target region from anatomical features of the patient. Alternatively, the computing system may define such boundaries by comparing the differences between the surgical pathway and/or the surgical target region from anatomical features within the representation.

During a surgical procedure, the computing system may track the position of a surgical instrument. If the surgical instrument comes into proximity with one of the defined surgical boundaries, the computing system may provide feedback to the surgeon. In some examples, the nature of the feedback may vary based on the anatomical feature on the other side of the defined boundary. For instance, a specific anatomical feature may be designated as critical or non-critical. If the computing system determines that the surgical instrument is in proximity with a non-critical feature, the computing system may provide a first level of feedback, such as a visual or audio alert. But, if the computing system determines that the surgical instrument is in proximity with a non-critical feature, the computing system may provide a second level of feedback, such as haptic feedback. In some cases, the computing system may even prevent the surgical instrument from crossing the surgical boundary. The nature of the feedback may also vary based on the distance between the surgical instrument and the boundary. For instance, the feedback may get more intense as the surgical instrument gets closer to the boundary. Other examples of feedback are possible as well.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 shows a simplified block diagram of a computing system, in accordance with an example embodiment.

FIG. 2 shows an illustrative computer-readable medium, in accordance with another example embodiment.

FIG. 3 shows an illustrative method providing feedback indicating that a surgical instrument within a threshold distance from a surgical boundary.

FIG. 4 shows an illustrative representation of the head of a patient.

FIG. 5 shows another illustrative representation of the head of a patient.

FIG. 6 shows yet another illustrative representation of the head of a patient.

FIG. 7 shows another illustrative representation of the head of a patient.

FIG. 8 shows illustrative approach vectors indicating surgical portals.

FIG. 9 shows a representation of an example surgical boundary and indication of a position of a surgical instrument.

### DETAILED DESCRIPTION

In the following detailed description, reference is made to the accompanying figures, which form a part thereof. In the figures, similar symbols typically identify similar components, unless context dictates otherwise. The illustrative examples described in the detailed description, figures, and claims are not meant to be limiting. Other examples may be utilized, and other changes may be made, without departing from the spirit or scope of the subject matter presented herein. It will be readily understood that aspects of the present disclosure, as generally described herein, and illustrated in the figures, can be arranged, substituted, combined, separated, and/or designed in a wide variety of different configurations, all of which are explicitly contemplated herein.

### 1. Example Architecture

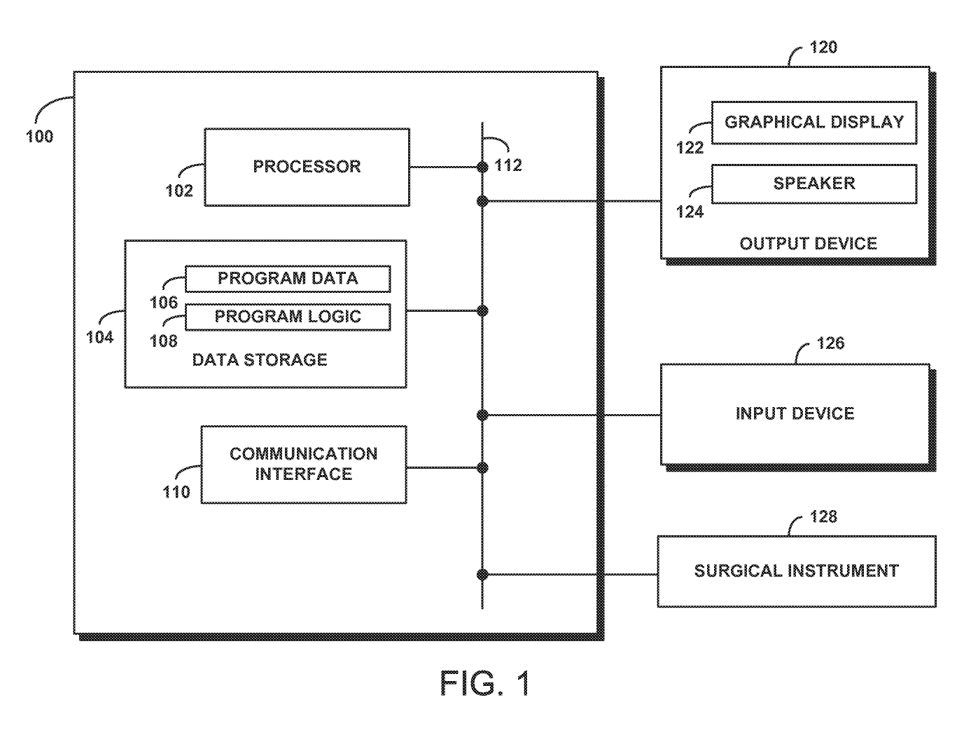

FIG. 1 shows a simplified block diagram of an example computing system 100 in which the present method can be implemented. It should be understood that this and other arrangements described herein are set forth only as examples. Those skilled in the art will appreciate that other arrangements and elements (e.g., machines, interfaces, functions, orders, and groupings of functions, etc.) can be used instead and that some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. And various functions described herein may be carried out by a processor executing instructions stored in memory.

As shown in FIG. 1, computing system 100 may include processor 102, data storage 104, and communication interface 110, all linked together via system bus, network, or other connection mechanism 112. Computing system 100 may be part of a surgical navigation system. Commercially-available surgical navigation systems include the STEALTHSTATION from MEDTRONIC and the NAVIGATION SYSTEM II from STRYKER, among many other examples.

Processor 102 may include one or more general purpose microprocessors and/or one or more dedicated signal processors and may be integrated in whole or in part with communication interface 110. Data storage 104 may include memory and/or other storage components, such as optical, magnetic, organic or other memory disc storage, which can be volatile and/or non-volatile, internal and/or external, and integrated in whole or in part with processor 102. Data storage 104 may be arranged to contain (i) program data 106 and (ii) program logic 108. Although these components are described herein as separate data storage elements, the elements could just as well be physically integrated together or distributed in various other ways. For example, program data 106 may be maintained in data storage 104 separate from program logic 108, for easy updating and reference by program logic 108.

Communication interface 110 typically functions to communicatively couple computing system 100 to networks. As such, communication interface 110 may include a wired (e.g., Ethernet) and/or wireless (e.g., Wi-Fi) packet-data interface, for communicating with other devices, entities, and/or networks. Computing system 100 may also include multiple interfaces 110, such as one through which computing system 100 sends communication, and one through which computing system 100 receives communication.

Computing system 100 may also include, or may be otherwise communicatively coupled to, output device 120. Output device 120 may include one or more elements for providing output, for example, one or more graphical displays 122 and/or a speaker 124. In operation, output device 120 may be configured to display a graphical user interface (GUI) via graphical display 122, corresponding to use of such a GUI.

Computing system 100 may further include, or may be otherwise communicatively coupled to, input device 126. Input device 126 may include one or more elements for receiving input, such as a keyboard and mouse. In some examples, input device 126 may include a touch-sensitive display, which may be incorporated into graphical display 122.

Computing system 100 may further be communicatively coupled to a surgical instrument 128. The surgical instrument may be any surgical instrument, such as an instrument for resection or an instrument for delivering therapeutics such as in chemotherapy or radiation therapy. The surgical instrument 128 may include a system that provides tracking of the position of the surgical instrument 128.

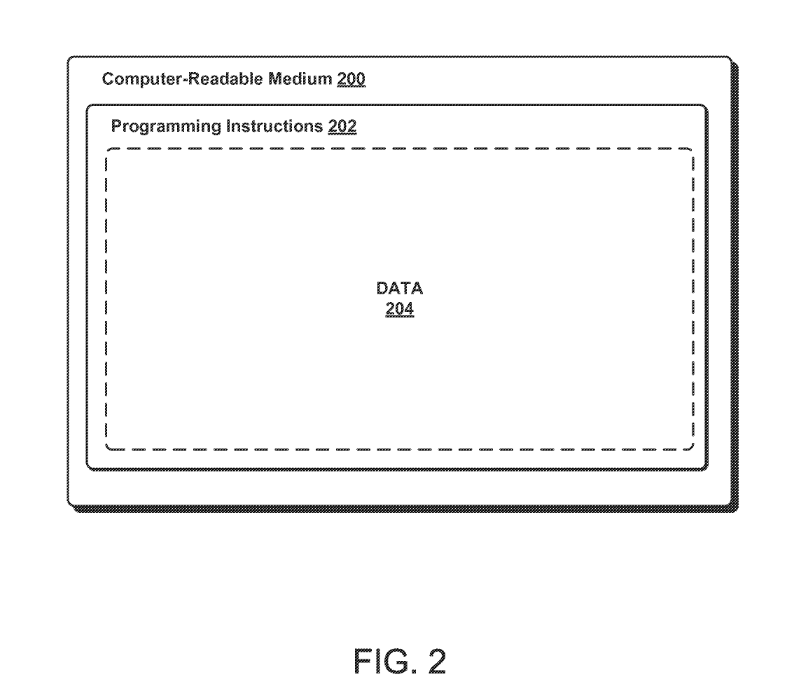

As noted above, in some examples, the disclosed methods may be implemented by computer program instructions encoded on a physical, and/or non-transitory, computer-readable storage media in a machine-readable format, or on other non-transitory media or articles of manufacture. FIG. 2 is a schematic illustrating a conceptual partial view of an example article of manufacture that includes a computer-readable medium for executing a computer process on a computing system, arranged according to at least some examples presented herein.

In one example, an example computer-readable medium 200 may include one or more programming instructions 202 that, when executed by one or more processors may provide functionality or portions of the functionality described herein. Example computer-readable mediums may include, but are not limited to, a hard disk drive, a Compact Disc (CD), a Digital Video Disk (DVD), a digital tape, memory, etc. In any event, computer-readable medium 200 is a physical, non-transitory, computer-readable medium. The programming instructions 202 may encompass data 204 included on the computer-readable medium 200.

### 2. Example Method

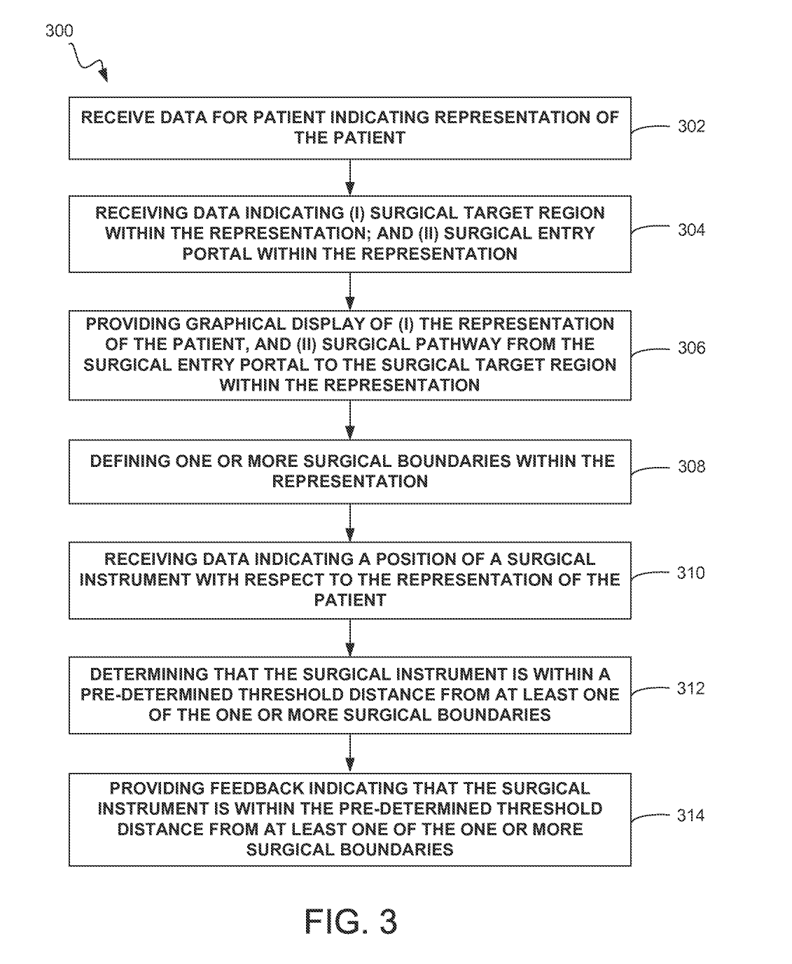

FIG. 3 shows a flowchart depicting functions that can be carried out in accordance with at least one embodiment of an example method. As shown in FIG. 3A, method 300 begins at block 302 with a computing system receiving, by a computing device, data for a patient indicating a representation of the patient. At block 304, the computing system receives data indicating (i) a surgical target region within the representation of the patient; and (ii) a surgical entry portal within the representation of the patient. At block 306, the computing system provides a graphical display of (i) the representation of the patient, and (ii) a surgical pathway from the surgical entry portal to the surgical target region within the representation. At block 308, for each surgical approach in the plurality of surgical approaches, the computing system, receives input defining one or more surgical boundaries within the representation. At block 310, the computing system receives data indicating a position of a surgical instrument with respect to the representation of the patient. At block 312, based on the received data indicating the position of the surgical instrument, the computing system determines that the surgical instrument is within a pre-determined threshold distance from at least one of the one or more surgical boundaries. At block 314, in response to the determining, the computing system, provides feedback indicating that the surgical instrument is within the pre-determined threshold distance from at least one of the one or more surgical boundaries.

In some implementations, method 300 may be carried out entirely, or in part, by computing system 100. Other suitable computing systems may be used as well.

a. Receiving Data for a Patient Indicating a Representation of the Patient

At block 302, the computing system 100 receives data for a patient indicating a representation of the patient. In some examples, the representation may be imaging data from a medical imaging machine, such as a magnetic resonance imaging (MRI) machine, a positron emission tomography (PET) machine, a computed tomography (CT) machine, or an X-ray machine. The representation may depict one or more anatomical features of the patient.

Computing system 100 in FIG. 1 may receive the data over the system bus, network, or other connection mechanism 112. In some examples, the computing system may receive the imaging data from another computing system via a network over communication interface 110. For instance, the computing system may receive the imaging data from a Digital Imaging and Communications in Medicine (DICOM) storage system. Alternatively, the computing system may receive the imaging data via a transfer from a data storage device, such as a hard disk drive or a USB flash drive. In other examples, the computing system may receive the imaging data via a transfer from a data storage medium, such as a CD-ROM disk. Many other examples are possible as well.

As noted above, the representation may be imaging data. The imaging data may include one or more images produced using one or more of a variety of medical imaging techniques such as MRI, PET, or CT. The imaging data may include images from different perspectives, such as sagittal, coronal, or transverse.

In some cases, the representation may be a three dimensional (3-d) representation. A 3-d representation may be provided from a set of medical images, known as a scan. For example, the computing system may combine multiple two-dimensional (2-d) images as layers to form a three-dimensional representation. In other examples, the medical imaging machine may produce a three-dimensional representation.

In some embodiments, the imaging data indicates signal intensity. Signal intensity may vary based on the density of the imaged subject matter. Different anatomical features within the representation may have different signal intensities, which appear in contrast (e.g., lighter or darker) on the image, thereby distinguishing the anatomical features. An image may have a pixel resolution, such as 512 pixels by 512 pixels, for a two-dimensional image. Or, where the image is 3-d, the image may have a voxel resolution, such as 512 voxels by 512 voxels by 66 voxels.

A pixel may represent a physical region within the image. For example, a pixel may represent a physical region of 0.8×0.8 mm. Therefore, the pixel is an approximation of that physical region. Likewise, a voxel may define a physical volume; for example, a volume of 0.8×0.8×7 mm. Because each pixel is an approximation of a physical region, each pixel may have a physical location. Such a physical location may be represented by a 2-d or 3-d coordinate system.

Each pixel in an image may have a signal intensity sample (signal intensity) associated with that respective pixel. The signal intensity associated with that respective pixel represents the amplitude of the signal at one point. However, it should be understood that a pixel is an approximation of a region. Therefore, the imaging data may be a 2-d or 3-d array of signal intensity data. Such an array may be referred to as an image matrix.

After receiving the representation, the computing system may define a coordinate system with respect to the representation. For instance, if the representation includes one or more two-dimensional (2-d) images, the computing system may define a 2-d coordinate system. Alternatively, the computing system may define a 3-d coordinate system. The origin of the coordinate system may be any point within the representation.

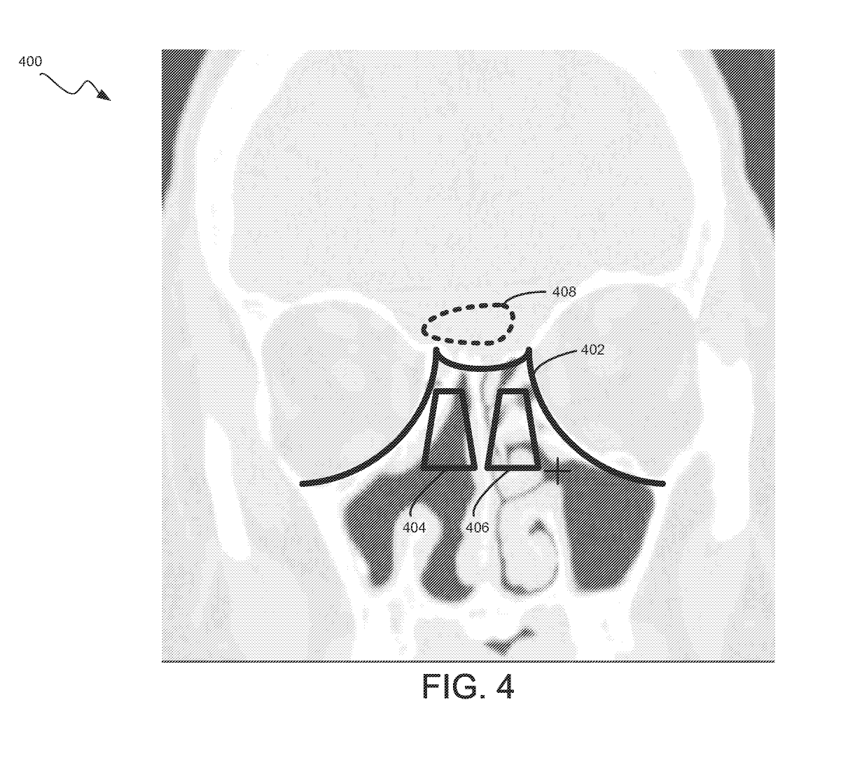

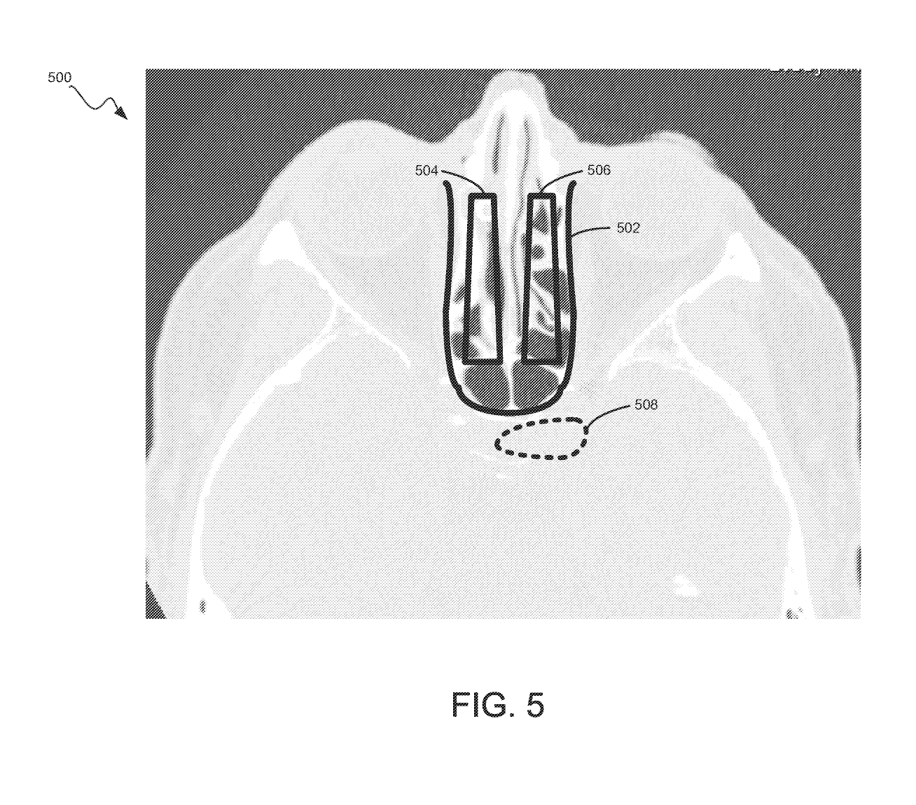

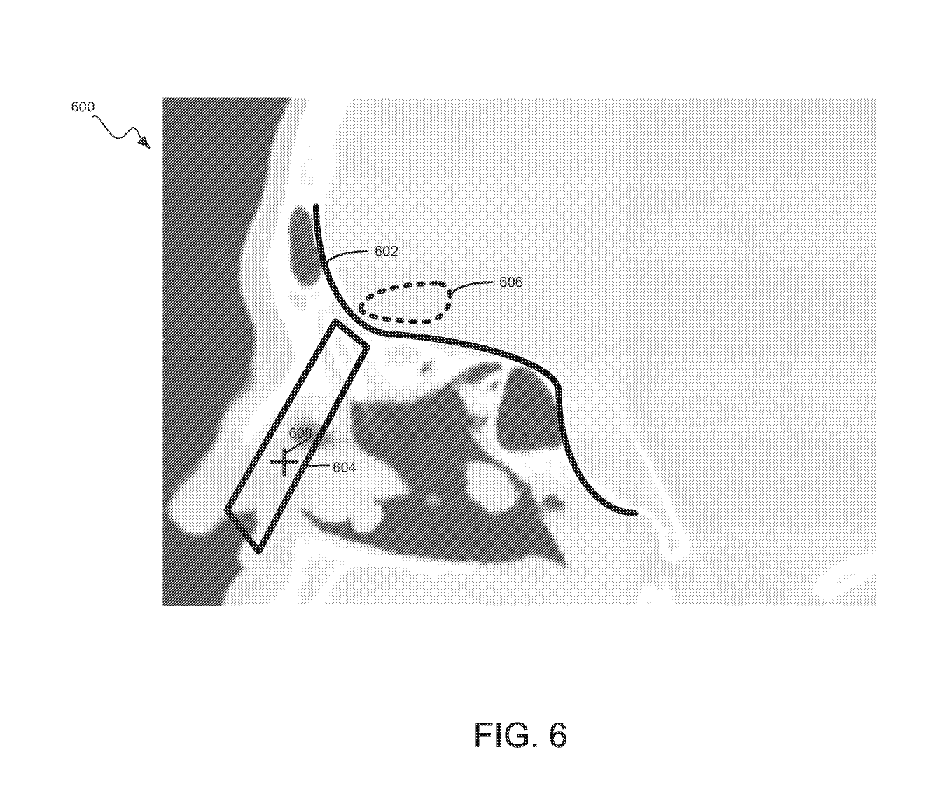

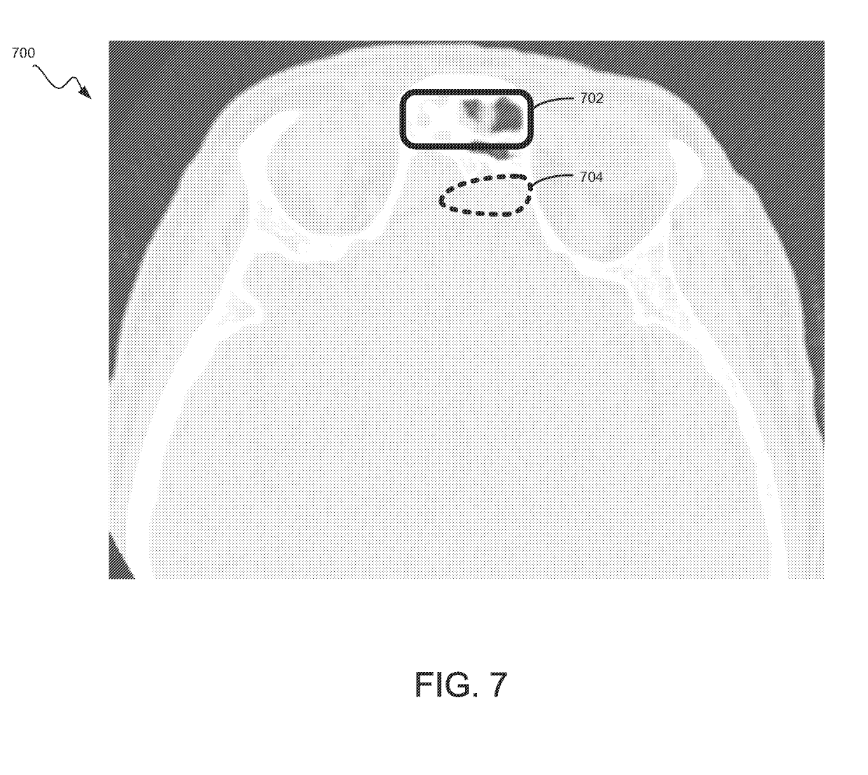

FIGS. 4-7 show illustrative representations of a head of a patient from different perspectives. FIG. 4 shows an illustrative representation 400. Representation 400 is a CT image depicting the head in the frontal plane. FIG. 5 shows another illustrative representation 500, which depicts the head in the axial plane. FIG. 6 shows yet another illustrative representation 600, which depicts the head in the sagittal plane. FIG. 7 shows another illustrative representation depicting the head in the axial plane. Although CT images are shown by way of example, images produced by other imaging techniques may be used as well.

Anatomical features of the patient depicted within the representation may then be respective volumes located at a set of points within the coordinate system. In some examples, the computing system may receive input designating certain areas as particular anatomical features. For instance, computing device may display the representation on graphical display 122. The surgeon may use the input device 126 to designate certain areas of the representation as particular anatomical features.

In other examples, the computing system may determine that areas with the representation depict certain anatomical features. Different anatomical features may have different signal intensities at one or more points, thereby creating a contrast between the anatomical features. The computing system may then segment the representation using any known or later discovered image segmentation technique, such as thresholding, clustering, edge detection, region-growing, and others. In some examples, the computing device may display an indication of such determined anatomical features on graphical display 122.

In some case, the computing device may display the determined anatomical features on the graphical display. The computing device may then receive input adjusting regions of the determined anatomical features. Such adjustment may correct errors in the segmentation.

b. Receiving Data Indicating (i) a Surgical Target Region within the Representation of the Patient, and (ii) a Surgical Pathway from the Surgical Entry Portal to the Surgical Target Region within the Representation

At block 304, the computing system receives data indicating (i) a surgical target region within the representation of the patient; and (ii) a surgical pathway from the surgical entry portal to the surgical target region within the representation.

The data indicating a surgical target region may define the surgical target area as a particular region within the representation. The surgical target region may be located at particular coordinates within the 2-d or 3-d coordinates system. In some cases, the computing system may receive the data from the input device 126. For instance, the computing device may display the representation on graphical device 122. The surgeon may then input the surgical target region via input device 126. Where the input device 126 includes a touch-sensitive display, the surgeon may draw the surgical target region on the touch-sensitive display. Other input techniques are possible as well. For example, the surgeon may use a pointing device, such as a mouse or trackpad, to input the surgical target region. Alternatively, the computing device may receive the data via communication interface 110.

The surgical target region may include a target pathology such as a lesion. In some circumstances, the target pathology may be located within an anatomical location. Such an anatomical location may include a cavity within the body. Alternatively, the anatomical location may include an anatomical feature. The surgical target region may define a 2-d or 3-d within the coordinate system. For example, the surgical target region may include a brain lesion, among many other possible examples. Conversely, the surgical target region may include one or more anatomical features that are not currently affected by pathology, but for which surgical manipulation is suggested for other reasons.

In some examples, the surgical target region may include a surgical margin. The surgical margin may define a region that fully or partially surrounds a target pathology that may be excised during the surgery. For example, the surgical margin may be an area of tumor free tissue surrounding a tumor that may be removed along with the tumor.

The one or more surgical target regions may be located within one or more of the following surgical target locations: pre-chiasmatic, post-chiasmatic, right cavernous sinus, left cavernous sinus, right Meckel's Cave, left Meckel's Cave, right superior orbital fissure, left superior orbital fissure, third ventricle extension, basal cistern extension, and clivus. The aforementioned example surgical target locations are located within the head. However, one having skill in the art will appreciate that many examples of surgical target regions in locations other than the head are possible. For example, surgical target regions within the chest or abdomen are possible. Moreover, surgical target regions at alternative locations within the head are possible as well.

In some examples, such a surgical target region may be manipulated by one or more surgical instruments. In the case where the surgical target region includes a target pathology, manipulation of the surgical target region may be performed to remove the pathology, for example, to remove a lesion. Manipulation of the surgical target region for removal of the lesion may include various techniques such as ablation. In other examples, one or more surgical instruments may deliver therapeutic agents to the surgical target region. For instance, the one or more surgical instruments may deliver radiation or chemo-therapy agents to the surgical target region. While manipulation of the target region may occur, manipulation of the surgical target region is not necessary to the method described herein.

FIGS. 4-7 illustrate example surgical target regions. FIGS. 4, 5, 6, and 7 include example surgical target regions 408, 508, 606, and 704, respectively.

The computing device may also receive or have access to data indicating one or more surgical portals. The data may define the one or more surgical portals as regions within the representation. Such regions may include particular data points that define the surgical portal within the 2-d or 3-d coordinate system.

The surgical portals may be entry points for surgical instrument into the human body. During a surgical procedure, surgical instruments may be inserted into the surgical portal. Some surgical portals may be openings, or orifices, into the human body that provide entry points that ease access into the body. The transnasal portal is an example of an opening that provides access into the skull, a part of the body. Other surgical portals may be entry points that ease access into the skull when some part of the human anatomy is displaced. For example, the transorbital and supraorbital portals provide access into the skull when the eye is displaced. Other surgical portals may be points at which incisions are made to provide access for surgical instruments. Certain surgical portals may be chosen for a particular surgery based on the relative location of the chosen surgical portal to the surgical target region.

For example, the one or more surgical portals may include one or more of the following surgical portals: right transnasal, left transnasal, right superior lid crease (superior orbit wall), right lateral retrocanthal (lateral orbit wall), right transconjuctival (inferior orbit wall), right precaruncular (medial orbital wall), left superior lid crease (superior orbital wall), left lateral retrocanthal (lateral orbit wall), left transconjunctival (inferior orbital wall), and left precaruncular (medial orbital wall). The aforementioned example surgical portals are located within the skull. However, one having skill in the art will appreciate that many examples of surgical portals in locations other than the skull are possible. For example, the surgical portals may be located on the exterior of the chest or abdomen. Or, as another example, the surgical portals may include the anus. Moreover, surgical portals at additional locations within the skull are possible as well.

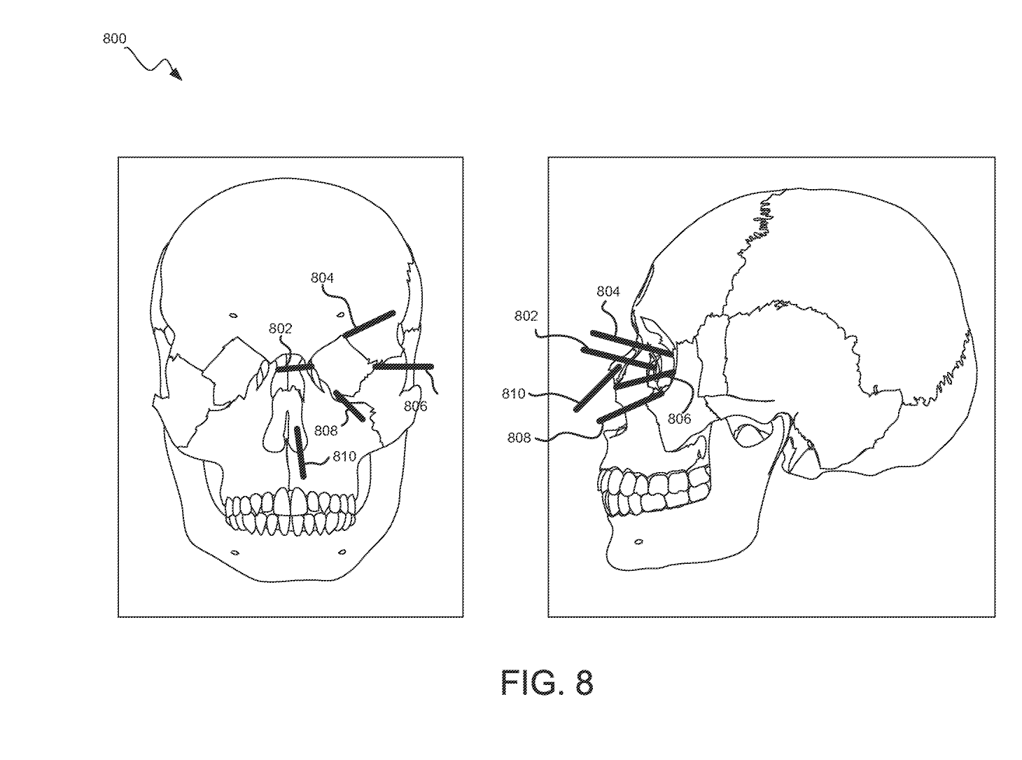

FIG. 8 depicts an illustrative model 800 that shows example surgical portals that are indicated by approach vectors 802, 804, 806, 808, and 810. The approach vectors may aid in visualization of the surgical portals. Each of approach vectors 802, 804, 806, 808, and 810 indicates a surgical portal at one end of the respective approach vector. Approach vector 802 indicates the left precaruncular portal. Approach vector 804 indicates the left superior lid crease portal. Approach vector 806 indicates the left lateral retrocanthal portal. Approach vector 808 indicates the left transconjunctival portal. And, approach vector 810 indicates the left transnasal portal. The approach vectors indicating surgical portals are provide for example only and should not be taken as limiting. Other surgical portals certainly exist but are not shown in this example.

c. Providing a Graphical Display of (i) the Representation of the Patient and (ii) a Surgical Pathway from the Surgical Entry Portal to the Surgical Target Region within the Representation

At block 306, the computing system provides a graphical display of (i) the representation of the patient and (ii) a surgical pathway from the surgical entry portal to the surgical target region within the representation. The computing system may provide the graphical display via output device 120. For instance, the computing system may cause graphical display 122 to display the representation of the patient and the surgical pathway.

The graphical display of the surgical pathway from the surgical entry portal to the surgical target region within the representation may represent the surgical pathway in different ways. For instance, the graphical display may represent the surgical pathway as a particular region within the representation. Alternatively, the graphical display may represent the surgical pathway as a path (e.g., a line) extending from the surgical entry portal to the surgical target region. Other forms of representation of the surgical pathway are possible as well. For instance, the graphical display may represent the surgical pathway as a 3-d volume. In some cases, the surgical pathway is referred to by way of the portal. In such a case, the graphical display may represent the surgical pathway by way of the portal. For instance, the graphical display may represent the surgical pathway as an approach vector intersecting the surgical portal at the angle of approach.

The graphical display may overlay such a region or a line over the representation of the patient. Such an overlay may show anatomical features of the patient in relation to the surgical pathway. The graphical display may also represent the surgical portal and/or the surgical target region on the graphical display.

d. Defining One or More Surgical Boundaries within the Representation

At block 308, the computing system defines one or more surgical boundaries within the representation. In some examples, after providing the graphical display, the computing system may receive input defining the one or more surgical boundaries within the representation. In other examples, based on the received data indicating the (i) surgical target region and (ii) surgical entry portal, the computing system may determine one or more surgical boundaries within the representation. The one or more surgical boundaries may divide the surgical target region and the surgical pathway from the one or more anatomical features of the patient. The data points within the coordinate system may represent the surgical boundaries.

As noted above, in some examples, the computing system may receive input defining the one or more surgical boundaries within the representation. For instance, the surgeon may review the graphical display of (i) the representation of the patient and (ii) the surgical pathway from the surgical entry portal to the surgical target region within the representation. The surgeon may then input the surgical boundaries via input device 126. As noted above, the input device may include a touch-sensitive display. In that case, the surgeon may input the surgical boundaries on the representation of the patient via input device 126 and graphical display 122. For example, the surgeon may draw the surgical boundaries on the representation of the patient. In other cases, the surgeon may input the surgical boundaries using any suitable input device, such as a keyboard and/or a mouse. Alternatively, the computing device may receive the input defining the one or more surgical boundaries within the representation via communication interface 110.

The surgical boundaries may divide the surgical pathway and/or the surgical target region from one or more anatomical features of the patient. For instance, in endoscopic sinus surgery, the surgical boundaries may divide the eyes from the transnasal pathways. In another example, if the surgical target region is a brain lesion, the surgical boundaries may divide the brain lesion from the surrounding brain tissue. As one with skill in the art will appreciate, in some circumstances, an anatomical feature such as skin or bone tissue may be removed from a surgical pathway to create or widen the pathway for the passage of surgical instruments. However, in other cases, the one or more anatomical features may be “critical” anatomical features for which transversal of the anatomical feature would cause unacceptable collateral damage. For example, in a surgical procedure involving a brain lesion, transversal of certain portions of the brain unaffected by the brain lesion may cause unacceptable collateral damage and so may be considered “critical.” The defined surgical boundaries may divide the surgical pathway from such “critical” anatomical features. While certain anatomical feature have been described as “critical” to facilitate comprehension of the described features, “critical” anatomical features are not necessary to the features.

In some cases, the computing system may divide the surgical boundaries into two or more levels (e.g., a first level and a second level). In some cases, a first level may represent a “suggested” surgical boundary. Transversal of the suggested surgical boundary may cause some collateral damage, but such collateral damage may be an acceptable aspect of the surgical procedure. The second level may represent a “required” surgical boundary. The “required” surgical boundary may divide the surgical pathway from “critical” anatomical features for which transversal of the anatomical feature would cause unacceptable collateral damage. Additional levels of surgical boundaries are possible as well. Such additional levels may represent varying degrees to which transversal of the boundary is acceptable or unacceptable.

In some cases, the computing system may receive data designating defined surgical boundaries as a particular level. For instance, the computing system may receive data designating at least one of the one or more surgical boundaries as one or more first surgical boundaries and at least one of the one or more surgical boundaries as one or more second surgical boundaries. From the surgeon's perspective, the surgeon may provide input designating at least one of the one or more surgical boundaries as one or more first surgical boundaries and at least one of the one or more surgical boundaries as one or more second surgical boundaries. In other cases, the computing system may recognize certain anatomical features via pattern matching or other object recognition techniques. The computing system may then designate certain anatomical features as particular levels based on data that indicates the respective level of each anatomical feature. In some examples, the computing system may use a combination of such approaches. Other techniques for designating levels of surgical boundaries are possible as well.

FIGS. 4-7 illustrate example surgical boundaries. FIG. 4 depicts example surgical boundaries 402, 404, and 406 within representation 400. The computing device may divide surgical boundaries 402, 404, and 406 into a first level and a second level. For instance, the computing system may receive input designating surgical boundaries 404 and 406 as a first level and surgical boundary 402 as a second level. Surgical boundary 402 divides the surgical pathway from the eyes, while surgical boundaries 404 and 406 divide the surgical pathway from less-critical skin and/or bone tissue. FIG. 5 depicts example surgical boundaries 502, 504, and 506. Like surgical boundaries 402, 404, and 406, the computing system may divide the surgical boundaries into one or more levels. For instance, the computing system may designate surgical boundaries 504 and 506 as a first level and surgical boundary 502 as a second level. FIG. 6 and FIG. 7 depict example surgical boundaries 602 and 604 and example surgical boundary 702, respectively.

As noted above, in some examples, the computing system may determine the one or more surgical boundaries within the representation based on the received data. For instance, the computing system may segment or otherwise divide certain anatomical features from the surgical pathway based on differences in the signal intensities between the anatomical features and the surgical pathway in the representation. The computing system may also segment or otherwise divide certain anatomical features from the surgical target region based on differences in the signal intensities between the anatomical features and the surgical target region in the representation. As noted above, the computing system may use any suitable image segmentation technique, such as thresholding, clustering, edge detection, region-growing, and others.

For example, the representation may include an image indicating signal intensities. An anatomical figure and the surgical target region may have first signal intensities and second signal intensities, respectively. Then, to determine the one or more surgical boundaries within the representation, the computing system may determine differences at one or more points in the image between the first signal intensities and the second signal intensities. The computing system may then define the one or more surgical boundaries as the line formed by interconnection of the one or more points to divide the one or more anatomical features from the surgical target region. Other examples are possible as well.

As noted above, surgical boundaries may have different levels, or be otherwise differentiated. In one example, the computing system designating at least one of the one or more anatomical features as a first anatomical feature and at least one of the one or more anatomical features as a second anatomical feature. Then, to determine the one or more surgical boundaries within the representation, the computing system may determine at least one first surgical boundary within the representation. The at least one first surgical boundary may divide the surgical target region and the surgical pathway from the first anatomical feature. The computing system may also determine at least one second surgical boundary within the representation. The at least one second surgical boundary may divide the surgical target region and the surgical pathway from the second anatomical feature. This technique may be repeated for additional anatomical features.

In some examples, the computing system may display an indication of the determined surgical boundaries on a graphical display, such as graphical display 122. In some cases, the surgeon may review such determined surgical boundaries on the graphical display and then provide input adjusting the determined surgical boundaries. The computing device may then receive input indicating one or more alterations to the determined one or more surgical boundaries. Such adjustment may correct errors in the segmentation. Based on the received input, the computing system may alter the determined one or more surgical boundaries, such as by moving the boundary from one position in the representation to another, or by altering the path of the boundary. Further, the graphical display of the determined surgical boundaries may facilitate navigation during a surgical procedure, among other possible benefits.

e. Receiving Data Indicating a Position of a Surgical Instrument with Respect to the Representation of the Patient

At block 310, the computing system receives data indicating a position of a surgical instrument with respect to the representation of the patient. As noted above, in one example, the computing device integrated into or communicatively coupled to a surgical navigation system. As noted above, example commercially available surgical navigation systems include the STEALTHSTATION from MEDTRONIC and the NAVIGATION SYSTEM II from STRYKER.

Such surgical navigations systems may include instrument tracking systems.

An example instrument tracking system may include a surgical instrument having one or more magnetic coils. In operation, the instrument tracking system may generate an electromagnetic field. The instrument tracking system may then track the electromagnetic field to triangulate the position of the magnetic coil as the magnetic coil affects the magnetic field. Other types of instrument tracking systems are possible as well.

During a surgical procedure, the computing system may track the position of the surgical instrument. For instance, the computing system may receive the position of the surgical instrument periodically, such as every 100 ms. The computing system may provide a graphical display indicating the position of the surgical instrument in relation to the representation on the graphical display. Further, the computing system may provide a graphical display indicating the position of the surgical instrument with respect to the one or more surgical boundaries within the representation. As the surgical instrument moves in relation to the representation, the computing system may update the display to indicate the new position.

The computing system may track the position of the instrument in relation to the coordinate system of the representation may represent the position of the instrument. The one or more coordinates within the representation. For instance, in the example instrument tracking system above, a set of coordinates may represent the position of the magnetic coil.

FIG. 608 shows an example indication 608 of the position of the surgical instrument with respect to the representation of the patient 608. Indication 608 may represent the point of the surgical instrument in which the magnetic coil, or other tracking means, is located, tip of the surgical instrument. For instance, indication 608 may represent the tip of the surgical instrument. Alternatively, indication 608 may represent some other point within the surgical instrument.

f. Determining that the Surgical Instrument is within a Pre-Determined Threshold Distance from at Least One of the One or More Surgical Boundaries

At block 312, the computing system determines that the surgical instrument is within a pre-determined threshold distance from at least one of the one or more surgical boundaries. The computing system may make the determination based on the received data indicating the position of the surgical instrument.

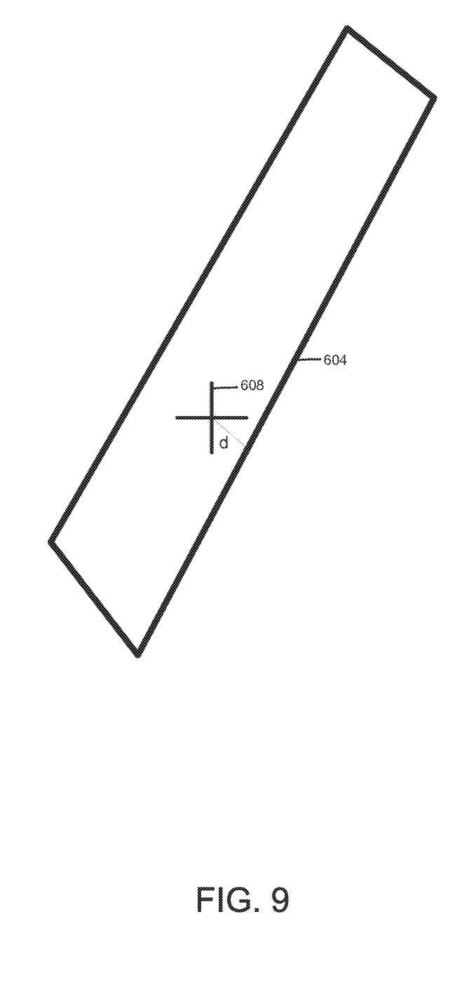

FIG. 9 is a simplified representation of surgical boundary 604 depicted in FIG. 6. FIG. 9 also shows indication 608 of the position of the surgical instrument. As noted above, the surgical boundary and the position of the surgical instrument may represent respective points or sets of points within the coordinate system. For instance, the indication 608 position of the surgical instrument may be (x₁, y₁) within representation 600. The surgical boundary 608 may similarly be located at a set of points [(x₂, y₂) . . . (xₙ, yₙ)]. The computing system may determine the distance ‘d’ as shown in FIG. 9 between the surgical instrument at (x₁, y₁) and the nearest point on the surgical boundary 608 located at the set of points [(x₂, y₂) . . . (xₙ, yₙ)].

Further, each point, or pixel, may represent a physical area or volume. Based on the physical area represented by each point, the computing system may translate the distance ‘d’ in the coordinate system between the surgical instrument at (x₁, y₁) and the nearest point on the surgical boundary 608 to a physical distance between the surgical instrument and the surgical boundary. The computing system may then determine whether the physical distance between the surgical instrument and the surgical boundary is less than the pre-determined threshold distance.

The pre-determined threshold distance may be pre-determined at different distances based on the patient and the surgical procedure being performed. Surgical procedures involving the head may suggest a relatively smaller threshold distance than surgical procedures involving the abdomen or chest. In some cases, the pre-determined threshold distance may be set at 0.5 millimeters (mm) or 1 mm. In other cases, the pre-determined threshold distance may be set at 0 mm.

As noted above, the computing system may track the position of the surgical instrument by receiving the position of the surgical instrument periodically, or at some other interval, such as when the position of the surgical instrument is changed. In some cases, the computing system may determine that the surgical instrument is within the pre-determined threshold distance after receiving the position of the surgical instrument. Alternatively, the computing system may determine that the surgical instrument is within the pre-determined threshold distance when the position of the surgical instrument is changed. In some examples, the computing system may determine that the surgical instrument is within the pre-determined threshold distance in response to receiving the position of the surgical instrument. Other examples are possible as well.

Also as noted above, the surgical boundaries may have different levels, or be otherwise differentiated (e.g., into first surgical boundaries and second surgical boundaries). In one example, the computing system may determine that the surgical instrument is within the first pre-determined threshold distance from the at least one of the one or more first surgical boundaries. The computing system may also determine that the surgical instrument is within the second pre-determined threshold distance from the at least one of the one or more second surgical boundaries. In some cases, the differentiated surgical boundaries may have different pre-determined threshold distances. For instance, a first pre-determined threshold distance (for the first surgical boundaries) may be 0 mm and a second pre-determined threshold distance (for the second surgical boundaries) may be 2 mm. Such different surgical boundaries may facilitate protecting “critical” anatomical features while also allowing the surgeon discretion during the surgical procedure.

g. Providing Feedback Indicating that the Surgical Instrument is within the Pre-Determined Threshold Distance from at Least One of the One or More Surgical Boundaries

At block 314, the computing device provides feedback indicating that the surgical instrument is within the pre-determined threshold distance from at least one of the one or more surgical boundaries. The computing system may provide feedback in response to determining that the surgical instrument is within a pre-determined threshold distance from at least one of the one or more surgical boundaries. The computing system may provide feedback in a variety of ways. In some examples, the feedback may be intended to catch the surgeon's attention by creating sensory stimulation. For example, the feedback may be a visual, audio, or haptic feedback. In some cases, the computing system may provide a combination of two or more different types of feedback.

As noted above, the computing system may provide visual feedback. For instance, the computing system may provide a graphical display an indication on graphical display 122 that the surgical instrument is within a pre-determined threshold distance from at least one of the one or more surgical boundaries. Alternatively, the computing system may cause a graphical display of a surgical navigation system to display an indication that the surgical instrument is within the pre-determined threshold distance from at least one of the one or more surgical boundaries.

The indication may be a message, an icon, or any suitable indication. The indication may include flashing the graphical display or a portion thereof. Alternatively, the computing system may cause a warning light or other visual indicator to turn on. Many examples of visual feedback are possible.

Alternatively, the computing system may provide audio feedback. For instance, the computing system may cause speaker 124 to emit an audio alert. The audio alert may be a buzzer or a tone, or it may be a more complex alert such as a pre-recorded voice message. Many examples of audio alerts are certainly possible.

In some cases, the computing system may provide haptic feedback. For instance, the computing system may cause the surgical instrument to vibrate. Such haptic feedback may provide feedback to the surgeon without necessitating that the surgeon divert his eyes from the patient or the surgical navigation.

In some examples, the feedback may vary in intensity. For instance, audio feedback may vary in volume, or haptic feedback may vary in intensity of vibration. In some examples, the computing system may provide feedback that is proportional in intensity to the distance between the surgical instrument and the nearest point to the surgical instrument along the one or more surgical boundaries. For instance, the computing system may cause an audible alert to sound at a sound intensity level that is proportional to the distance between the surgical instrument and the point along the one or more surgical boundaries. In one example, the audio alert may get louder in volume as the surgical instrument becomes nearer to a surgical boundary. In another example, the computing system may cause the surgical instrument to vibrate at a intensity level that is proportional to the distance between the surgical instrument and the point along the one or more surgical boundaries. In an example, the vibration may become more intense as the surgical instrument becomes nearer to the surgical boundary.

In some examples, the surgical instrument may operate via electrical power. For instance, the surgical instrument may be an electrically-powered cutting tool, such as a drill or saw. With an electrically-powered surgical instrument, the computing system may provide feedback by causing the surgical instrument to cease operation, such as by disconnecting the electrical power supply to the electrically-powered tool. Such feedback may assist in preventing collateral damage to the patient in the event that one of the one or more surgical boundaries are crossed by the surgical instrument.

Further, the surgical instrument may be a robotic instrument coupled to a robotic arm. The robotic arm may move the surgical instrument according to controls from a control system. The surgical instrument may be an end-effector of the robotic arm. With a robotic surgical instrument, the computing system may provide feedback by causing the robotic arm to move the surgical instrument. For example, the computing system may cause the robotic arm to move the surgical instrument to move to a point within the one or more surgical boundaries. For instance, the computing system may cause the robotic arm to move the surgical instrument to a point within the one or more surgical boundaries and nearest the point of intersection with one of the one or more surgical boundaries. Alternatively, the computing system may cause the robotic arm to center the surgical instrument within the surgical pathway, or within the surgical target region. Other examples are possible as well.

As noted above, the computing system may designate certain surgical boundaries as different levels (e.g., first and second boundaries). In response to determining that the surgical instrument is within a threshold distance of a particular surgical boundary, the computing system may provide a different level of feedback depending on the level of the particular surgical boundary. For example, in response to determining that the surgical instrument is within the first pre-determined threshold distance, the computing system may provide a first level of feedback indicating that the surgical instrument is within a first pre-determined threshold distance from at least one of the one or more first surgical boundaries. In response to determining that the surgical instrument is within the second pre-determined threshold distance, the computing system may provide a second level of feedback indicating that the surgical instrument is within a second pre-determined threshold distance from at least one of the one or more second surgical boundaries.

As noted above, the first and second levels of surgical boundary may represent “suggested” and “required” surgical boundaries, respectively. The first and second levels of feedback may vary in intensity. For example, the first level of feedback may be sensory feedback, such as haptic, audible, or visual feedback. Such sensory feedback may assist in notifying the surgeon that he or she is near a first-level boundary but allow the surgeon the discretion to ignore the feedback. As noted above, in some cases, some collateral damage that may result from crossing a first-level boundary may be acceptable to complete the surgical procedure. The second level of feedback may physically prevent the surgical instrument from intersecting a second-level boundary. For example, the computing device may cause the surgical instrument to cease operation or to move within the surgical boundary. Such physical prevention may assist in protecting “critical” anatomical features from damage.

In some examples, the computing system may prevent the surgical instrument from intersecting the one or more surgical boundaries. This feature may cause the surgical boundary to function as a “lock-in” or “lock-out” zone. In effect, the surgical boundary may “lock-in” the surgical instrument to the surgical pathway and/or the surgical target region and “lock-out” the surgical instrument from anatomical features on the other side of the surgical boundary. Such functionality may facilitate aspects of surgical procedures. For instance, an example surgical procedure may involves resection of the surgical target region. To resect the surgical target region, the surgeon may move a surgical instrument within the surgical target region to remove tissue within the region. But, the surgeon must be careful not to move the surgical instrument outside of the surgical target region, or collateral damage may result. If the surgical boundary “locks-in” the surgical instrument, the surgeon may use the surgical boundary as a guide during the surgical procedure to prevent the surgical instrument from traversing outside of the surgical target region.

In some cases, the surgical procedure may involve two or more surgical instruments, such as a first surgical instrument and a second surgical instrument. The computing system may provide different feedback depending on whether the first surgical instrument or the second surgical instrument is within the pre-defined threshold distance from the one or more surgical boundaries. In some cases, the computing system may provide feedback when the first surgical instrument and the second surgical instrument are within different pre-defined threshold distances, such as a first pre-defined threshold distance and a second pre-defined threshold distance, respectively. For instance, the first surgical instrument may be a drill, and the first pre-defined threshold distance may be 2 mm. The second surgical instrument may be an endoscope and the second pre-defined threshold distance may be 0 mm. Many combinations of surgical instruments and pre-defined threshold distances are possible.

### 3. Conclusion

While various aspects and examples have been disclosed herein, other aspects and examples will be apparent to those skilled in the art. For example, with respect to the flow charts depicted in the figures and discussed herein, functions described as blocks may be executed out of order from that shown or discussed, including substantially concurrent or in reverse order, depending on the functionality involved. Further, more or fewer blocks and/or functions may be used and/or flow charts may be combined with one another, in part or in whole.

The various aspects and examples disclosed herein are for purposes of illustration and are not intended to be limiting, with the true scope and spirit being indicated by the following claims. Other examples can be utilized, and other changes can be made, without departing from the spirit or scope of the subject matter presented herein.

1. A computer-implemented method comprising:
receiving, by a computing device, data for a patient indicating a representation of the patient;
receiving data indicating (i) a surgical target region within the representation of the patient; and (ii) a surgical entry portal within the representation of the patient;
providing a graphical display of (i) the representation of the patient, and (ii) a surgical pathway from the surgical entry portal to the surgical target region within the representation;
defining one or more surgical boundaries within the representation;
receiving data indicating a position of a surgical instrument with respect to the representation of the patient;
based on the received data indicating the position of the surgical instrument, determining that the surgical instrument is within a pre-determined threshold distance from at least one of the one or more surgical boundaries; and
in response to the determining, providing feedback indicating that the surgical instrument is within the pre-determined threshold distance from at least one of the one or more surgical boundaries.

2. The computer-implemented method of claim 1, further comprising:
providing a graphical display indicating the position of the surgical instrument with respect to the one or more surgical boundaries within the representation.

3. The computer-implemented method of claim 1, wherein providing feedback comprises:
causing a graphical display of a surgical navigation system to display an indication that the surgical instrument is within the pre-determined threshold distance from at least one of the one or more surgical boundaries.

4. The computer-implemented method of claim 1, further comprising:
preventing the surgical instrument from intersecting the one or more surgical boundaries.

5. The computer-implemented method of claim 1, wherein providing feedback comprises:
providing haptic feedback to the surgical instrument.

6. The computer-implemented method of claim 1, further comprising:
determining a distance between the surgical instrument and a point nearest to the surgical instrument along the one or more surgical boundaries; and
wherein providing feedback comprises:
causing an audible alert to sound at a particular sound intensity level, wherein the sound intensity level is proportional to the distance between the surgical instrument and the point.

7. The computer-implemented method of claim 1, further comprising:
determining a distance between the surgical instrument and a point nearest to the surgical instrument along the one or more surgical boundaries; and
wherein providing feedback comprises:
causing the surgical instrument to vibrate at a particular intensity level, wherein the particular intensity level is proportional to the distance between the surgical instrument and the point.

8. The computer-implemented method of claim 1, wherein the surgical instrument comprises a robotic surgical instrument, and wherein providing feedback comprises:
causing the robotic surgical instrument to move to a point within the one or more surgical boundaries.

9. The computer-implemented method of claim 1, wherein providing feedback comprises:
causing the surgical instrument to cease operation.

10. The computer-implemented method of claim 1, further comprising:
receiving data designating at least one of the one or more surgical boundaries as one or more first surgical boundaries and at least one of the one or more surgical boundaries as one or more second surgical boundaries; and
wherein determining that the surgical instrument is within the pre-determined threshold distance from the at least one of the one or more surgical boundaries comprises:
determining that the surgical instrument is within the first pre-determined threshold distance from the at least one of the one or more first surgical boundaries; and;
determining that the surgical instrument is within the second pre-determined threshold distance from the at least one of the one or more second surgical boundaries.

11. The computer-implemented method of claim 10, wherein providing feedback comprises:
in response to determining that the surgical instrument is within the first pre-determined threshold distance, providing a first level of feedback indicating that the surgical instrument is within a first pre-determined threshold distance from at least one of the one or more first surgical boundaries; and
in response to determining that the surgical instrument is within the second pre-determined threshold distance, providing a second level of feedback indicating that the surgical instrument is within a second pre-determined threshold distance from at least one of the one or more second surgical boundaries.

12. The computer-implemented method of claim 1, wherein defining one or more surgical boundaries within the representation comprises:
after providing the graphical display, receiving input defining one or more surgical boundaries within the representation.

13. The computer-implemented method of claim 1, wherein defining one or more surgical boundaries within the representation comprises:
based on the received data indicating the (i) surgical target region and (ii) surgical entry portal, determining one or more surgical boundaries within the representation, wherein the one or more surgical boundaries divide the surgical target region and the surgical pathway from the one or more anatomical features of the patient.

14. The computer-implemented method of claim 13, wherein the representation comprises an image indicating first signal intensities of the one or more anatomical features and second signal intensities of the surgical target region, and wherein determining one or more surgical boundaries within the representation comprises:
determining differences at one or more points in the image between the first signal intensities and the second signal intensities; and
defining the one or more surgical boundaries as the line formed by interconnection of the one or more points to divide the one or more anatomical features from the surgical target region.

15. The computer-implemented method of claim 13, further comprising:
receiving data designating at least one of the one or more anatomical features as a first anatomical feature and at least one of the one or more anatomical features as a second anatomical feature; wherein determining the one or more surgical boundaries within the representation comprises:
determining at least one first surgical boundary within the representation, wherein the at least one first surgical boundary divides the surgical target region and the surgical pathway from the first anatomical feature; and
determining at least one second surgical boundary within the representation, wherein the at least one second surgical boundary divides the surgical target region and the surgical pathway from the second anatomical feature.

16. The computer-implemented method of claim 13, further comprising:
receiving input indicating one or more alterations to the determined one or more surgical boundaries; and
based on the received input, altering the determined one or more surgical boundaries.

17. The computer-implemented method of claim 1, wherein receiving data indicating a position of the surgical instrument with respect to the representation of the patient comprises tracking the position of the surgical instrument during a surgical procedure.

18. A computing system comprising:
a physical computer readable medium; and
program instructions stored on the physical computer readable medium and executable by at least one processor to:
receive, by a computing device, data for a patient indicating a representation of the patient;
receive data indicating (i) a surgical target region within the representation of the patient; and (ii) a surgical entry portal within the representation of the patient;
provide a graphical display of (i) the representation of the patient, and (ii) a surgical pathway from the surgical entry portal to the surgical target region within the representation;
define one or more surgical boundaries within the representation;
receive data indicating a position of a surgical instrument with respect to the representation of the patient;
based on the received data indicating the position of the surgical instrument, determine that the surgical instrument is within a pre-determined threshold distance from at least one of the one or more surgical boundaries; and
in response to the determining, provide feedback indicating that the surgical instrument is within the pre-determined threshold distance from at least one of the one or more surgical boundaries.

19.-34. (canceled)

35. An article of manufacture comprising a non-transitory tangible computer readable medium configured to store at least executable instructions, wherein the executable instructions, when executed by a processor of a computing device, cause the computing device to perform functions comprising:
receiving, by a computing device, data for a patient indicating a representation of the patient;
receiving data indicating (i) a surgical target region within the representation of the patient; and (ii) a surgical entry portal within the representation of the patient;
providing a graphical display of (i) the representation of the patient, and (ii) a surgical pathway from the surgical entry portal to the surgical target region within the representation; defining one or more surgical boundaries within the representation;
receiving data indicating a position of a surgical instrument with respect to the representation of the patient;
based on the received data indicating the position of the surgical instrument, determining that the surgical instrument is within a pre-determined threshold distance from at least one of the one or more surgical boundaries; and
in response to the determining, providing feedback indicating that the surgical instrument is within the pre-determined threshold distance from at least one of the one or more surgical boundaries.

36.-51. (canceled)
